(19) United States
(12) Patent Application Publication
(10) Pub. No.: US 2010/0152635 A1
(43) Pub. Date: Jun. 17, 2010

(54) MAGNETIC ARM SLING

(76) Inventor: Peter S. Borden, Palos Verdes Estates, CA (US)

Correspondence Address: DICKSTEIN SHAPIRO LLP, 1825 EYE STREET NW, Washington, DC, 20006-5403, US

(21) Appl. No.: 12/635,441

(22) Filed: Dec. 10, 2009

Related U.S. Application Data
(60) Provisional application No. 61/122,630, filed on Dec. 15, 2008.

(51) Int. Cl. A61F 5/00

(52) U.S. Cl. 602/4

(57) ABSTRACT

A magnetic arm sling for immobilizing the arm of a patient. The magnetic arm sling includes an abdominal strap with a magnetically attractive member and an arm support with a magnetically attractive member. The arm support is worn on the patient's arm. The magnetically attractive member of the abdominal strap and the magnetically attractive member of the arm support form a magnetic bond to immobilizes the patient's arm. An abduction pillow with one or more magnetically attractive members may also be used with the magnetic arm sling.

This application claims the benefit of U.S. Provisional Application No. 61/122,630, filed on Dec. 15, 2008, the entire disclosure of which is incorporated by reference herein.

### BACKGROUND OF THE INVENTION

1. Field of the Invention

The invention relates to an arm sling for supporting an injured arm or shoulder and, more specifically, to an arm sling which uses magnets to immobilize the arm in a desired position.

2. Description of the Related Art

Various types of arm slings are known for use in supporting an arm during the healing process in the treatment of injuries to the shoulder or the arm and wrist. Slings are used to hold the shoulder, arm, or wrist in place and to restrict movement by the shoulder, arm, or wrist such that healing occurs. Simple slings comprise a pouch and a strap connected to the pouch, wherein the strap loops around the neck of the patient to support the pouch. The forearm of the patient rests in the pouch. U.S. Pat. Nos. 4,372,301, 4,622,961, and 4,834,082 are examples of these types of simple shoulder slings. Additionally, an arm-sling vest can be worn to promote healing, as shown in U.S. Pat. No. 6,453,904.

Slings have evolved over the years to not only provide the function of supporting an immobilized arm, but also to provide abduction. U.S. Pat. Nos. 7,563,236 and 4,598,701 disclose slings which provide abduction.

In a typical arm sling, the neck of the patient supports all of the weight of the supported arm, often causing discomfort to the patient. The strap may also rub the patient's neck, adding to the patient's discomfort. Because the entire arm of the patient is confined in the typical arm sling, these slings place unwanted pressure against the neck and elbow areas causing secondary problems to the extremity such as: increased neck pain, nerve compression at the elbow causing hand numbness, and skin irritation from the strap. Additionally, these known arm slings immobile the patient's entire arm, from the shoulder to the patient's wrist, even if immobilization of the entire arm is unnecessary. Finally, these past arm slings must all be worn completely over the clothing of the wearer.

Therefore, a need exists for a shoulder or arm sling which can be used to treat injuries by restricting movement and providing abduction, while at the same time providing the patient with a more comfortable experience, immobilizing only the necessary body parts, and with the ability to be worn discretely.

### SUMMARY OF THE INVENTION

The present invention is a magnetic arm sling for use in treatment of the arm or shoulder. The magnetic arm sling comprises an abdominal strap and an arm support. The abdominal belt includes at least one magnetically attractive member. The arm support also includes at least one magnetically attractive member. The arm support is worn on the arm of the patient. The magnetically attractive members of the abdominal strap and the arm support form a magnetic bond. The magnetic bond immobilizes the patient's arm in a manner to prevent movement and promote healing. By utilizing a magnetic bond, the magnetic arm sling of the present invention can be completely or partially worn under the clothing of the patient.

A first embodiment of the invention utilizes an abdominal strap and two arm supports, an upper arm support and a forearm support. The upper arm support and the forearm support magnetically bond to the abdominal strap at two different positions, immobilizing the forearm and upper arm of the patient.

A second embodiment utilizes an abdominal strap and an upper arm support. The upper arm support magnetically bonds to the abdominal strap in a manner such that the upper arm of the patient is immobilized to the side of the patient's body, leaving the forearm and hand free to move.

A third embodiment utilizes an abdominal strap, an upper arm support, a forearm support, and an abduction pillow. The abduction pillow includes a magnetically attractive member which magnetically bonds to the abduction belt. The forearm support then magnetically bonds to the abdominal strap. The upper arm support magnetically bonds to the abdominal strap in a manner such that the upper arm of the patient upper arm is immobilized to the side of the patient's body.

The various embodiments of the invention advantageously provide support for an arm or shoulder of a patient without placing extra stress on the patient's neck and back. Furthermore, no pressure is exerted upon the elbow, because the elbow is not confined. Only the portions of the arm which need to be immobilized are immobilized. Finally, the patent can wear the magnetic sling system underneath his or her clothing.

### BRIEF DESCRIPTION OF THE DRAWINGS

FIG. 1a is a front view of the abdominal strap component of the magnetic arm sling system.

FIG. 1b is a front view of the upper arm support of the magnetic arm sling system.

FIG. 1c is a front view of the upper arm support of the magnetic arm sling system.

FIG. 2 is a top view of a first embodiment of the magnetic arm sling as worn by a patient.

FIG. 3 is a front view of a first embodiment of the magnetic arm sling as worn by a patent.

FIG. 4 is a top view of a second embodiment of the magnetic arm sling as worn by a patient.

FIG. 5a is a top view of an abduction pillow.

FIG. 5b is a front view of an abduction pillow.

FIG. 6 is a top view of a third embodiment of the magnetic arm sling as worn by a patient.

### DETAILED DESCRIPTION OF THE PREFERRED EMBODIMENTS

The following description is provided to enable any person skilled in the art to make and use the invention and sets forth the best modes contemplated by the inventors of carrying out their inventions. Various modifications, however, will remain readily apparent to those skilled in the art.

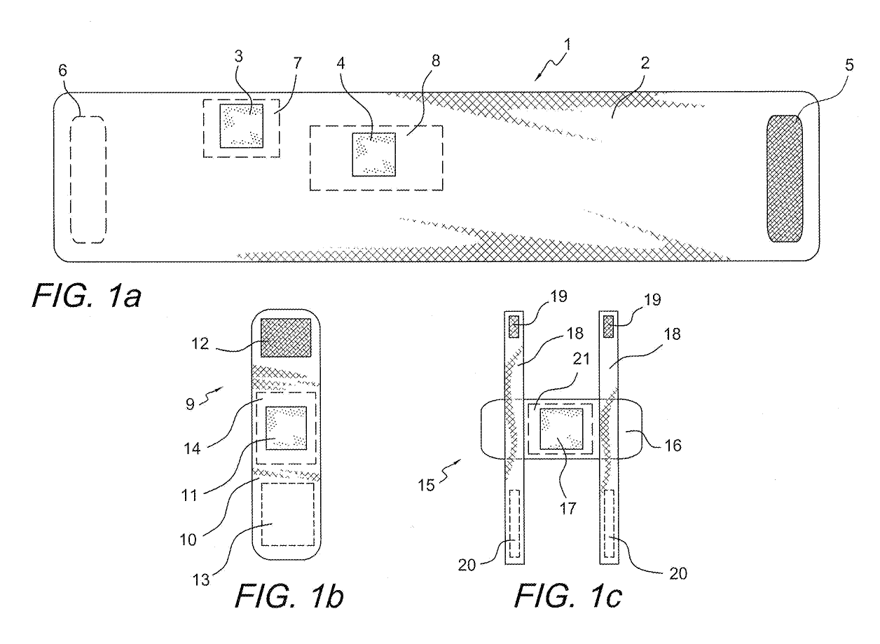

FIGS. 1a-1c provide a front-view of various components of the magnetic arm sling of the present invention. FIG. 1a shows an abdominal strap 1, which comprises a body member 2 that is wrapped around the abdomen of the patient. In the described embodiment, after being fitted around the abdomen of the patient, the body member 2 of the abdominal strap 1 is secured to itself. The abdominal strap is secured by fastening the ends of the body member 2 to each other. This can be accomplished, for example, by using complimentary hook and loop fasteners (such as Velcro™), such that the first end of the body member includes hook fasteners 5 on the front of the body member and the second end of the body member includes loop fasteners 6 on the back of the body member. It is possible to use other fastening means, such as snaps, buttons/button holes, zippers, lacing, etc. The abdominal strap also includes magnetically attractive members 3 and 4. The magnetically attractive members 3 and 4 are preferably inserted into respective pouches 7 and 8 within the body member 2 or pouches 7 and 8 attached to the surface of the body member 2. Alternatively, the magnetically attractive members 3 and 4 can be attached directly on the outer surface of the body member 2. The magnetically attractive members 3 and 4 can comprise, for example, permanent magnets, electromagnets, or materials capable of forming a magnetic bond to a magnet.

FIG. 1b shows an upper arm support 9 which may be used with the abdominal strap 1. The upper arm support 9 comprises a body member 10 which is wrapped around the upper arm (the portion of the arm from the elbow to the shoulder) of the patient. After being wrapped around the upper arm of the patient, the body member 10 of the upper arm support 9 is secured in position by fastening the ends of the body member 10 to each other, for example using Velcro™, as described above with respect to abdominal strap 1. Again, it is possible to use other fastening means, such as snaps, buttons/button holes, zippers, lacing, etc. The upper arm support also comprises a magnetically attractive member 11. The magnetically attractive member 11 can be inserted into a pouch 14 within the upper arm support body member 10, inserted into a pouch 14 attached to the surface of the body member 10, or attached directly on the outer surface of the body member 10. The magnetically attractive member 11 can be a permanent magnet, electromagnet, or any material capable of forming a magnetic bond to a magnet.

FIG. 1c shows a forearm support 15 which may be used with the abdominal strap 1. The forearm support 15 includes a padded body member 16, which is placed on the underside of the forearm of a patent. The padded body member 16 may be various lengths, depending on the patient's body size and needs, and can extend from the elbow to across the wrist joint. The padded body member 16 can comprise a variety of soft materials, for example, memory foam. While comfort against the forearm is important, it may be desirable that the padded body member 16 also provide firmness across the wrist joint. This can be accomplished by using a rigid base for body member 16, and including padding on top of the rigid base 16. The forearm support 15 can be secured to the underside of the patient's forearm (the portion of the arm from the elbow to the wrist) using securing straps 18. The ends of the securing straps 18 can be fastened to each other to secure the forearm support 15 to the patient's forearm, using Velcro™ or other fastening means, such as snaps, buttons/button holes, zippers, lacing, etc., as described above. The forearm support 15 also includes a magnetically attractive member 17 inserted into a pouch 21 within the padded body member 16, inserted into a pouch 21 attached to the surface of the body member 16, or attached directly on the outer surface of the body member 16. The magnetically attractive member 17 can be, for example, a permanent magnet, electro magnet, or a material capable of forming a magnetic bond to a magnet.

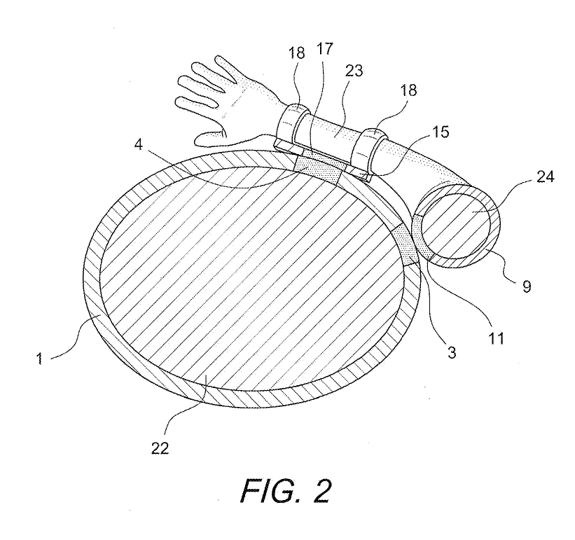

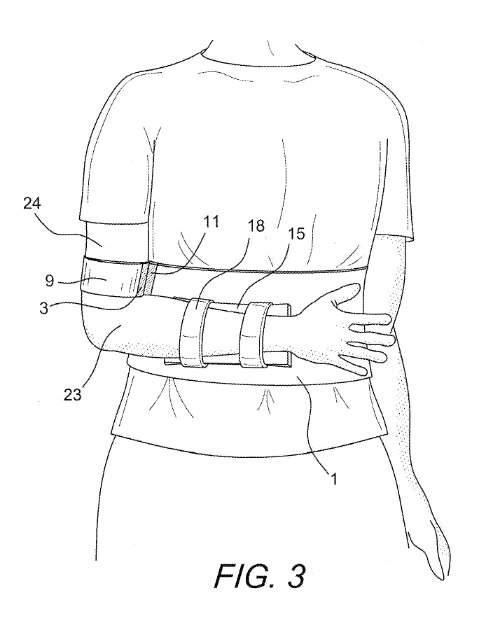

FIGS. 2 and 3 show a first embodiment of the magnetic arm sling system as worn by a patient to treat the right arm, comprising the abdominal strap 1, the upper arm support 9, and the forearm support 15 as worn by a patient for treatment of the right arm or shoulder. FIG. 2 is a top view of the magnetic arm sling system. FIG. 3 is a front-view of the magnetic arm sling system. As shown, the abdominal strap 1 is fitted around the abdomen of the patient 22. The patient wears the upper arm support 9 around his or her upper arm 24. The patient wears forearm support 15 around his or her forearm 23, attached to the underside of the forearm by securing straps 18.

As shown, the magnetically attractive member 4 of the abdominal strap 1 is located at a position complimentary to the magnetically attractive member 17 of forearm strap 15, such that a magnetic bond forms between magnetically attractive members 4 and 17, securing the forearm to a fixed position. The magnetically attractive member 3 of the abdominal strap 1 is complimentary to the magnetically attractive member 11 of the arm strap 9, such that a magnetic bond forms between those magnetically attractive members 3 and 11, securing the upper arm to a fixed position. The position of the abdominal strap 1 and its magnetically attractive members 3 and 4 can be adjusted in a manner which positions the arm to best promote healing, and to best fit the body of the patient. For example, the position of magnetically attractive members 3 and 4 can be adjusted longitudinally or laterally within their respective pouches 7 and 8 such the patent's arm is immobilized in a desired position. Likewise, magnetically attractive members 11 of upper arm support 9, and magnetically attractive member 17 of forearm support 15, can be repositioned within their respective pouches 14 and 21. Generally, magnetically attractive member 3 is positioned higher than magnetically attractive member 4, because the patient's upper arm is immobilized at his or her side, and the patient's forearm is immobilized around the patient's abdomen.

Magnetically attractive members 3 and 4 are generally laterally spaced so that the magnetically attractive member 17 of forearm support 15 is disposed in a position corresponding to magnetically attractive member 4 and magnetically attractive member 11 of upper arm support 9 is disposed in a position corresponding to magnetically attractive member 3. Advantageously, the magnetic bond between the magnetically attractive members 3 and 11, and 4 and 17 does not have to be direct; i.e. because magnets are used, there can be a magnetic bond between the abdominal strap 1 and the upper arm support 9, and abdominal strap 1 and forearm support 15 through the clothing of the patient. In this manner, the patient can wear the magnetic arm sling system completely, or partially underneath the patient's clothing.

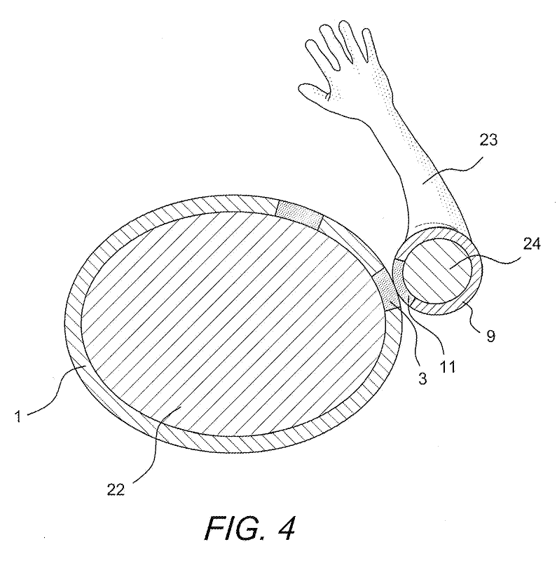

FIG. 4 shows a top view of a second embodiment of the magnetic arm sling system, where only the upper arm of the patient must be immobilized, making use of the abdominal strap 1 and the upper arm support 9 as worn by a patient for treatment of the right arm or shoulder. As shown, the abdominal strap 1 is fitted around the abdomen of the patient 22. The patient wears the upper arm support 9 around his or her upper arm 24.

As shown, magnetically attractive member 3 of the abdominal strap 1 is located in a position complimentary to the magnetically attractive member 9 of the arm strap 9, such that a magnetic bond forms between magnetically attractive members 3 and 11, securing the upper arm in a fixed position. Again, the position of the abdominal strap 1 and its magnetically attractive member 3 can be adjusted longitudinally or laterally to best promote patient healing and fit.

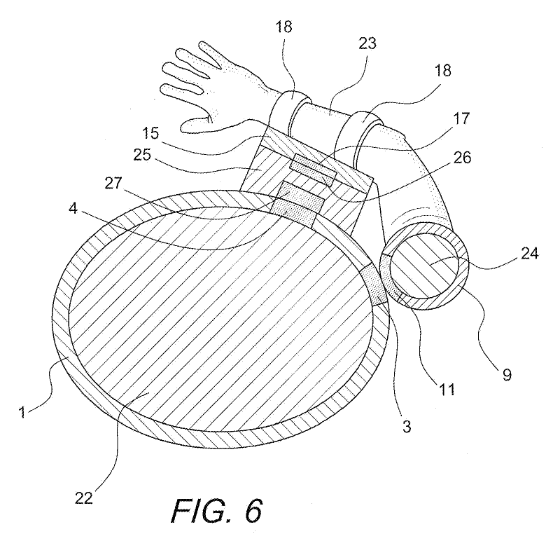

FIG. 6 is a top view of a third embodiment of the magnetic arm sling system, making use of the abdominal strap 1, the upper arm support 9, the forearm support 15, and an abduction pillow 25 as worn by a patient for treatment of the right arm or shoulder. As shown, the abdominal strap 1 is fitted around the abdomen of the patient 22. The patient wears the upper arm support 9 around his or her upper arm 24. The patient also wears forearm support 15 around his or her forearm 23, attached to the underside of the forearm by supporting straps 18. Additionally, an abduction pillow 25 between the abdomen 22 and forearm 23 to provide abduction for the patient's arm.

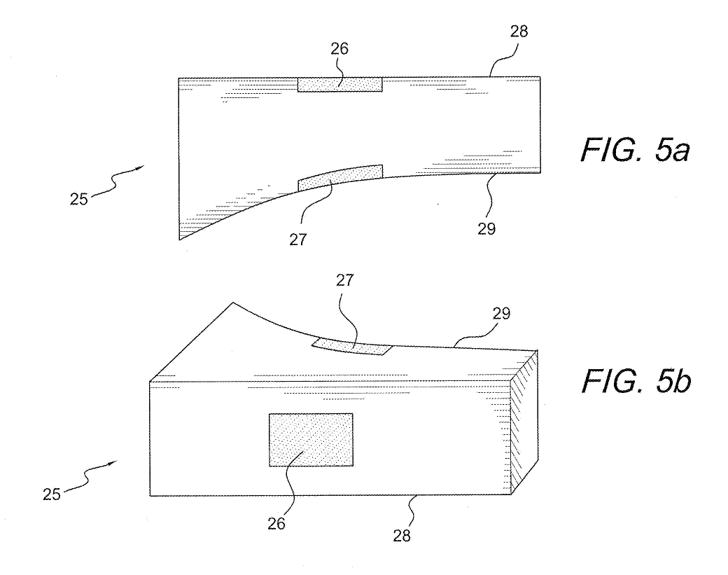

FIGS. 5a and 5b show a top and front view of abduction pillow 25. The abduction pillow includes magnetically attractive members 26 and 27. Magnetically attractive member 27 is located near the back end 29 of the abduction pillow 25. Magnetically attractive member 26 is located towards the front end 28 of the abduction pillow 25. As shown, the abduction pillow's back end 29 can be contoured to fit against the body 22 of the patient.

Referring again to FIG. 6, the magnetically attractive member 3 of the abdominal strap 1 is located in a position complimentary to the magnetically attractive member 11 of the arm strap 9, and the magnetically attractive member 4 of the abdominal strap 1 is located in a position complimentary to the magnetically attractive member 27 of abduction pillow 22, such that a magnetic bond forms between those magnetically attractive members 11 and 9, and between magnetically attractive members 4 and 27, thereby securing the back end of the abduction pillow 29 to the patient's body 22. The magnetically attractive member 26 of the abduction pillow 25 is located in a position complimentary to the magnetically attractive member 17 of forearm strap 15, such that a magnetic bond forms between those magnetically attractive members 17 and 26, thus securing the patient's forearm 23 to the abduction pillow 25. Thus, immobilization and abduction for the treatment of the arm is provided.

As described above with respect to other embodiments of the invention, the positions of the abdominal strap 1 and its magnetically attractive members 3 and 4 can be adjusted longitudinally or laterally in a manner which best promotes healing, and which best fits the body of the patient. Generally, magnetically attractive member 3 is positioned higher than magnetically attractive member 4, because the patient's upper arm is immobilized at his or her side, and the patient's forearm is immobilized at the level of the patient's abdomen. As stated above, the magnetic bond between the magnetically attractive members 3 and 11, 4 and 27, and 17 and 26 does not have to be direct; i.e. because magnets are used, there can be a bond between the abdominal strap 1 and the abduction pillow 25, or the abduction pillow 25 and the forearm support 15, through the clothing of the patient. Thus, as in the other embodiments, the patient can wear the magnetic arm sling system completely, or partially underneath his or her clothing.

As discussed above, the magnetically attractive members may be a magnet, electromagnet, or material which is attracted to the magnetic field of a magnet. However, it is clear that for a magnetic bond to exist between magnetically attractive members, there must be a magnetic field existing between the members. Therefore, where magnetic bonds are described, a magnetic field must exist. A magnetic field can be produced by, for example, a permanent magnet or electromagnet. One or both magnetically attractive members can be a magnet. Furthermore, none of the magnetically attractive members of the arm sling must be a magnet; a magnet can be disposed between two magnetically attractive members (which do not provide a magnetic field) to magnetically bond the members.

A magnetically attractive member may also comprise multiple smaller magnetically attractive members. For example, a collection of magnetically attractive members may be used instead of a larger, single magnetically attractive member.

The strength of the magnetic bond by the magnetically attractive members of the abdominal strap 1, the upper arm support 9, the forearm support 15, or the abduction pillow 25 can be adjusted by using magnets of different strength. The strength of the magnetic bond strength may be adjusted for various reasons, such as to match the weight of the patient's arm, or to compensate for the weakening magnetic force if the magnetic bond between components must penetrate through layers of material. It is important to adjust the magnetic bond strength to provide enough strength to secure the arm in a healing position. Magnetic bond strength, related to magnetic field strength, can be adjusted in many well-known ways. For example, various types of permanent magnets exist, with different magnetic field strengths, such as ferrite, alnico, neodymium, or samarium-cobalt magnets. If electromagnets are employed, the strength of the electromagnet can be controlled by adjusting the level of current applied. Providing more or less magnets, or varying the shape and size of the magnetic field will also adjust the magnetic bond strength.

In summary, the present invention provides a magnetic arm sling system to immobilize a patient's arm or shoulder without using the patient's neck and shoulder to support the arm, without confining or putting pressure on the elbow. The invention allows immobilization of only necessary members of a patient's arm, allow greater use of the patient's arm. The invention also provides the patient the option of wearing the magnetic arm sling underneath clothing, allowing for more discrete use.

Those skilled in the art will appreciate that adoptions and modifications of the just-described preferred embodiment can be configured without departing from the scope and spirit of the invention. While the drawings show a magnetic arm sling for the patent's right-arm, the sling of the present invention can obviously be configured for left-arm use as well. Furthermore, the magnetically attractive members described may comprise multiple, smaller magnetically attractive members forming the larger magnetically attractive member; i.e. a magnetically attractive member does not need to be a single, continuous member. For example, a plurality of spaced magnets may comprise one magnetically attractive member.

What is claimed as new and desired to be protected by Letters Patent of the United States is:

1. An arm sling, comprising:
an abdominal strap including a first magnetically attractive member; and
a first arm support including a second magnetically attractive member;
wherein the first magnetically attractive member and the second magnetic are attracted to one another by a magnetic field such that the arm of a patient supported by the first arm support is immobilized.

2. The arm sling as recited in claim 1, wherein the abdominal strap further comprising a third magnetically attractive member which forms a magnetic bond with a fourth magnetically attractive member of a second arm support to further immobilize the arm of the patient.

3. The arm sling as recited in claim 2, further comprising:
an abduction pillow comprising:
a front end including a fifth magnetically attractive member;
a back end including a sixth magnetically attractive member;
wherein the third magnetically attractive member of the abdominal strap is magnetically bonded to the fifth magnetically attractive member of the abduction pillow and the fourth magnetically attractive member of the second arm support is magnetically bonded to the sixth magnetically attractive member of the abduction pillow.

4. The arm sling as recited in clam 1, wherein the first arm support is secured to a patient's upper arm.

5. The arm sling as recited in claim 2, wherein the first arm support is secured to a patient's upper arm and the second arm support is secured to a patient's forearm.

6. The arm sling as recited in claim 3, wherein the first arm support is secured to a patient's upper arm and the second arm support is secured to a patient's forearm.

7. The arm sling as recited in claim 1, wherein the first and second magnetically attractive members are magnets.

8. The arm sling as recited in claim 2, wherein the first, second, third and fourth magnetically attractive members are magnets.

9. The arm sling as recited in claim 3, wherein the first, second, third, fourth, fifth and sixth magnetically attractive members are magnets.

10. The arm sling as recited in claim 2, wherein the second arm support is padded.

11. The arm sling as recited in claim 1, wherein the first and second magnetically attractive members are selected from the group consisting of a magnet, an electromagnet, and a material that is attracted to the magnetic field of a magnet.

12. The arm sling as recited in claim 11, wherein both the first and second magnetically attractive members comprise magnets.

13. The arm sling as recited in claim 11, where only one the first and second magnetically attractive members comprises a magnet, and the other comprises a material that is attracted to a magnet.
